(19) United States
(12) Patent Application Publication
(10) Pub. No.: US 2024/0034991 A1
(43) Pub. Date: Feb. 1, 2024

(54) METHOD FOR EFFICIENTLY INDUCING REPROGRAMMING OF HUMAN CELL INTO NEURONAL CELL

(71) Applicants: GUANGXI UNIVERSITY, Nanning (CN); Guangxi Benpeng Biotechnology Co., Ltd., Nanning (CN)

(72) Inventors: Ben HUANG, Nanning (CN); Guodong WANG, Nanning (CN); Dandan ZHANG, Nanning (CN); Quanhui LIU, Nanning (CN); Yulian WU, Nanning (CN); Danwei LV, Nanning (CN); Jigang HU, Nanning (CN); Xiaolian XIE, Nanning (CN)

(73) Assignees: GUANGXI UNIVERSITY, Nanning (CN); Guangxi Benpeng Biotechnology Co., Ltd., Nanning (CN)

(21) Appl. No.: 17/874,433

(22) Filed: Jul. 27, 2022

(51) Int. Cl. C12N 5/0793

(52) U.S. Cl. CPC C12N 5/0619; C12N 2501/727; C12N 2501/115; C12N 2501/16; C12N 2501/999

(57) ABSTRACT

In a method for efficiently inducing reprogramming of human cells into neuronal cells, a concentration of cAMP is increased or an expression of PKA and CREB is up-regulated or an expression of AMPK, ALK2, ALK3, P38, and JNK is inhibited by a single small molecule compound or gene knockout or gene overexpression. In the present disclosure, the induction small molecule compound is single and safe, and has a short induction time, high induction efficiency, definite induction sites and genes, and clear molecular regulation mechanism. The small molecule compound can be applied to the clinical treatment of human neurodegenerative diseases, providing a safer and more efficient treatment method for the neurodegenerative diseases. Since neuronal cells cannot divide and proliferate, the method induces fibroblasts and astrocytes that can divide and proliferate in vitro and in vivo, to continuously obtain a large number of induced neuronal cells in vitro and in vivo.

### TECHNICAL FIELD

The present disclosure belongs to the field of stem cells, and in particular relates to a method for efficiently inducing reprogramming of human cells into neuronal cells.

### BACKGROUND ART

With induction methods such as transfection of exogenous transcription factors and combination of small molecule compounds, the reprogramming of terminally-differentiated somatic cells into neuronal cells has been achieved in human beings, mice and other species. In 2015, Pei Gang's group reported use of seven small molecule compounds in reprogramming fibroblasts from Alzheimer's disease patients into neuronal cells. In 2005, Brueckner B et al. used a combination of 6 small molecule compounds to induce human astrocytes into neuronal cells. These small molecule compounds inhibit the expression of non-neuronal genes, promote the expression of neuronal-specific genes, and eventually obtain mature functional neuronal cells through prolonged culture of induced cells.

Although there are many methods for inducing neuronal cells, these methods each have shortcomings such as long induction time (20 d to 30 d), low induction efficiency (10% to 30%), overly-cumbersome process, and high potential biosafety risks. In terms of cell therapy uses, the existing technology may greatly reduce an effect of clinical treatment due to use of excessive induction factors, overly long induction time, and relatively low efficiency, increasing potential treatment side effects and concerns about biological safety. In terms of mechanism, the existing technology is a combined action of various small molecule compounds, with intertwined signaling pathways of action and complicated action sites, which cannot accurately reveal how the fate transformation of somatic cells to neuronal cells is determined. At present, there is no technology that can use a single small molecule compound to reprogram somatic cells into neuronal cells, and the mechanism has not been fully elucidated in reprogramming of somatic cells into neuronal cells.

However, the induction system of the present disclosure can reprogram somatic cells into TUJ1-positive neuronal cells within two days (with a maximum positive rate of about 80%) under the premise of using only a single small molecule compound or a single gene for regulation. More importantly, the previously used methods of combining small molecule compounds cannot elucidate the mechanism of cell fate transformation. The discovery of the present disclosure clearly clarifies a molecular regulation path of reprogramming human cells into neuronal cells, and confirms that the regulation effect of a single small molecule compound or a single gene can induce the reprogramming of human cells into neuronal cells. In addition, the key regulatory sites for reprogramming mentioned in the present disclosure have not been reported.

### SUMMARY

In view of this, the present disclosure aims to propose a method for efficiently inducing reprogramming of human cells into neuronal cells, to overcome the defects of the prior art. The method confirms that the regulation effect of a single small molecule compound or a single gene can induce the reprogramming of human cells into neuronal cells.

To achieve the above objective, the present disclosure adopts the following technical solutions.

The present disclosure provides a method for efficiently inducing reprogramming of human cells into neuronal cells, including: increasing a concentration of cyclic Adenosine Monophosphate (cAMP), or up-regulating an expression of any site of protein kinase A (PKA) and Cyclic AMP response-element binding protein (CREB), or inhibiting an expression of any site of Adenosine 5′-monophosphate (AMP)-activated protein kinase (AMPK), activin receptor-like kinase 2 (ALK2), activin like kinase 3(ALK3), p38 mitogen-activated protein kinases (P38), and c-Jun N-terminal kinase (JNK).

Preferably, the “increasing a concentration of cAMP, or up-regulating an expression of any site of PKA and CREB, or inhibiting an expression of any site of AMPK, ALK2, ALK3, P38, and JNK” may be conducted by a small molecule compound, gene interference, gene knockout, or gene overexpression; preferably, the small molecule compound may include one or more of a cAMP activator, cAMP, a cAMP analog, a PKA activator, a CREB activator, an AMPK inhibitor, an ALK2 inhibitor, an ALK3 inhibitor, a P38 inhibitor, and a JNK inhibitor.

There are action sites inducing the reprogramming of human cells into neuronal cells and small molecule compounds that act on these sites. The action sites include cAMP (increasing concentrations), PKA (activating/up-regulating expressions), CREB (activating/up-regulating expressions), ALK2/3 (inhibiting/down-regulating expressions), JNK (inhibiting/down-regulating expressions), P38 (inhibiting/down-regulating expressions), and AMPK (inhibiting/down-regulating expressions). The single small molecule compounds include (but are not limited to) the followings: cAMP/PKA/CREB activators (Forskolin/Colforsin/8-Bromo-cAMP/Dibutyryl-cAMP (Bucladesine)/cAMP and analogs thereof), ALK2/3 inhibitors (such as LDN-193189/LDN-193189-2HCl/K02288/LDN-212854/LDN-214117/ML347/DMH 1), a JNK inhibitor (such as SP600125/Resveratrol/JNK-IN-8/JNK-Inhibitor VIII/DB07268/1Q-1S/Bentamapimod (AS602801)/Tanzisertib (CC-930)/BI-78D3/JNK Inhibitor IX/Urolithin B/Loureirin B/Loureirin B/Falcarindiol/Cucurbitacin IIb/Mulberroside A/Trans-Zeatin Astragaloside IV), a P38 inhibitor (such as SB203580/Doramapimod (BIRB796)/SB202190 (FHPI)/Ralimetinib dimesylate/VX-702/PH-797804/VX-745/TAK-715/PD169316/TA-02/SD0006/Pamap imod/BMS-582949/SB239063/Losmapimod (GW856553X)/Skepinone-L/SEA0400/AUDA/Praeruptorin A/Mulberroside A/UM-164/Trans-Zeatin/3′-Hydroxypterostilbene/Pexmetinib (ARRY-614), and an AMPK inhibitor (such as Dorsomorphin/Dorsomorphin (Compound C)/Dorsomorphin (Compound C) 2HCl/WZ4003/ON123300/HTH-01-015/Doxorubicin (Adriamycin)HCUGSK690693/XMD-17-51). These reagents each can induce the reprogramming of somatic cells such as human skin fibroblasts, human granulosa cells, and human astrocytes into functional neuronal cells.

The present disclosure further provides an induction medium for efficiently inducing reprogramming of human cells into neuronal cells, including a basal solution, KnockOut Serum Replacement (KSR), and a small molecule compound, where preferably, the small molecule compound includes one or more of a cAMP activator, cAMP, a cAMP analog (such as DBcAMP and 8-Cl-cAMP), a PKA activator, a CREB activator, an AMPK inhibitor, an ALK2 inhibitor, an ALK3 inhibitor, a P38 inhibitor, and a JNK inhibitor.

Preferably, the small molecule compound may include Forskolin, 8-Bromo-cAMP, LDN193189, the cAMP, the cAMP analog, SP600125, SB203580, and Dorsomorphin, with concentrations in a final medium sequentially as follows: 0 μM to 100 μM, 0 μM to 500 μM, 0 μM to 25 μM, 0 nM to 10 mM, 0 mM to 10 mM, 0 μM to 10 μM, 0 μM to 5 μM, and 0 μM to 100 μM, respectively, preferably 5 μM to 20 μM, 5 μM to 50 μM, 0.5 μM to 5 μM, 0.5 mM to 5 mM, 0.5 mM to 5 mM, 0.5 μM to 5 μM, 0.1 μM to 2.5 μM, and 0.5 μM to 20 μM, respectively, more preferably 10 μM, 50 μM, 2.5 μM, 1 mM, 1 mM, 1 μM, 0.5 μM, and 10 μM, respectively; and the concentration of the above substances may not be all 0.

Preferably, the basal solution and the KSR may have a volume ratio of 80:20; and preferably, the basal solution may be N2B27, including Knockout Dulbecco's Modified Eagle Medium: F-12 (DMEM/F12), N-2 Supplement (N2, 100×), Neurobasal, B-27 Supplement (B27, 50×), and Glutamine (100×) with a volume ratio of 99:1:97:2:1.

The present disclosure further provides use of the induction medium in in-vitro and in-vivo induction of reprogramming of somatic cells into neuronal cells.

The present disclosure further provides a method for inducing reprogramming of somatic cells into neuronal cells in vitro using an induction medium, including the following steps:
1) inoculating the somatic cells into a cell culture dish; adding a high-glucose dulbecco's modified eagle medium and 10% fetal bovine serum medium (DMEM+10% FBS), and conducting culture overnight in an incubator at 37° C. and a humidity of 95% with 5% carbon dioxide; conducting induction culture with the induction medium according to any one of claims 3 to 5 for 48 h to obtain chemical-induced neuronal cells (CiNCs); and
2) replacing the induction medium with a neuronal cell maturation medium to continue promoting further maturation of the CiNCs, and replacing the neuronal cell maturation medium with a neuronal cell medium after 72 h for long-term culture.

Preferably, the neuronal cell maturation medium may include DMEM/F12 and Neurobasal in a volume ratio of 1:1, 0.5% N2 (by volume percentage), 1% B27 (by volume percentage), 100 μM cAMP, 20 ng/mL basic fibroblast growth factor (bFGF), 20 ng/mL brain-derived neurotrophic factor (BDNF), 20 ng/mL glialcellline-derivedneurotrophicfactor (GDNF), 20 ng/mL Neurotrophin 3 (NT3), 100 U/mL penicillin, and 0.1 mg/mL streptomycin.

Preferably, the neuronal cell medium may include DMEM/F12 and Neurobasal in a volume ratio of 1:1, 0.5% N2 (by volume percentage), 1% B27 (by volume percentage), 20 ng/mL bFGF, 20 ng/mL BDNF, 20 ng/mL GDNF, 20 ng/mL NT3, 100 U/mL penicillin, and 0.1 mg/mL streptomycin.

Preferably, the somatic cells may be skin fibroblasts, granulosa cells, or astrocytes that are derived from a human being, a monkey, or a mouse.

Compared with the prior art, the method for efficiently inducing reprogramming of human cells into neuronal cells according to the present disclosure has the following beneficial effects:
(1) The present disclosure fills a gap in inducing the reprogramming of terminally-differentiated human cells into functional neuronal cells using a single small molecule compound.
(2) The present disclosure fills a gap in regulating the expression of a single gene (PKA, CREB, and JNK) to induce the reprogramming of terminally-differentiated human cells into functional neuronal cells.
(3) In the present disclosure, under the condition of using a single small molecule compound, neuronal cells with a beta-tubulin III (TUJ1) positive rate of about 80% can be obtained in only about two days of induction time. Compared with previous work, the induction time is greatly shortened and the induction efficiency is greatly improved.
(4) The present disclosure utilizes the characteristics of clear action pathways and clear action sites of a single small molecule compound, to clarify that the molecular regulation pathway of a whole reprogramming process of the somatic cells into the neuronal cells is cAMP-PKA-CREB-JNK and key regulatory genes thereof are PKA, CREB, and JNK. This mechanism has not yet been clearly elucidated.
(5) In the present disclosure, the induction small molecule compound is single and safe, and has a short induction time, high induction efficiency, and clear mechanism. The small molecule compound can be applied to the clinical treatment of human neurodegenerative diseases, providing a safer and more efficient treatment method for the neurodegenerative diseases. With the provided method, fibroblasts and astrocytes can be induced in vitro and in vivo, to continuously obtain a large number of induced neuronal cells in vivo and in vitro, thereby achieving the clinical treatment of neurodegenerative diseases using these regenerated neuronal cells.

### BRIEF DESCRIPTION OF THE DRAWINGS

As a part of the present disclosure, the accompanying drawings of the specification provide further understanding of the present disclosure. The schematic embodiments of the present disclosure and description thereof are intended to explain the present disclosure and are not intended to constitute an improper limitation to the present disclosure. In the accompanying drawings:

FIG. 1 shows a time pathway of a single small molecule compound Forskolin of Example 1 inducing the reprogramming of human skin fibroblasts into neuronal cells;

FIG. 2 shows a morphological change process of the single small molecule compound Forskolin of Example 1 inducing the reprogramming of human skin fibroblasts into neuronal cells;

FIG. 3 shows a immunofluorescence result of the single small molecule compound Forskolin of Example 1 inducing the reprogramming of human skin fibroblasts into neuronal cells;

FIG. 4 shows a quantitative polymerase chain reaction (PCR) result of the single small molecule compound Forskolin of Example 1 inducing the reprogramming of human skin fibroblasts into neuronal cells; and

FIG. 5 shows an influence of different single sites on a reprogramming regulatory pathway by an induction method of a small molecule compound, gene overexpression, or gene knockout.

### DETAILED DESCRIPTION OF THE EMBODIMENTS

Unless otherwise defined, the technical and scientific terms used in the following examples have the same meanings as commonly understood by those skilled in the art to which the present disclosure belongs. Unless otherwise specified, in the following examples, the test reagents used are all conventional biochemical reagents, and the test methods are all conventional methods.

The present disclosure will be described in detail below with reference to the accompanying drawings and the examples.

The present disclosure provides a method for efficiently inducing reprogramming of human cells into neuronal cells, including: increasing a concentration of cAMP, or up-regulating an expression of any site of PKA and CREB, or inhibiting an expression of any site of AMPK, ALK2, ALK3, P38, and JNK by a small molecule compound, gene interference, gene knockout, or gene overexpression; where the small molecule compound includes one or more of a cAMP activator, cAMP, a cAMP analog, a PKA activator, a CREB activator, an AMPK inhibitor, an ALK2 inhibitor, an ALK3 inhibitor, a P38 inhibitor, and a JNK inhibitor.
1. An induction medium of small molecule compounds used to regulate any site of a molecular pathway (increasing a concentration of cAMP, or up-regulating an expression of any site of PKA and CREB, or inhibiting an expression of any site of AMPK, ALK2, ALK3, P38, and JNK) of neuronal cell reprogramming (cAMP, PKA, CREB, AMPK, ALK2, ALK3, P38, and JNK) includes the following components:

| a basal solution (N2B27): a 200 mL system: | | |
|---|---|---|
| Knockout DMEM/F12 | 99 | mL |
| N2 (100×) | 1 | mL |
| Neurobasal | 97 | mL |
| B27 (50×) | 2 | mL |
| Glutamine (100×) | 1 | mL |
| an induction medium: a 100 mL system: | | |
| N2B27 | 80 | mL |
| KSR (serum replacer) | 20 | mL |

small molecule compounds name and concentration shown in Table 1 and Table 2.

TABLE 1
Induction concentration and efficiency of representative small molecule
compounds at each action site and gene verification of action site

| | | Name and | | | |
|---|---|---|---|---|---|
| | | concentration range | | | |
| | | of representative | | | Cell |
| | | small molecule | Induction | Neuron-specific | positive |
| Treatment | Action site | compounds | time | protein | rate |
| Small molecule | Activating | Forskolin (cAMP | 2 d | TUJ1 | 70%-80% |
| compound | AC (adenylate | activator): 1 μM to | | (beta-tubulin III) | |
| induction | activating | 100 μM | 2 d | MAP2 | 50%-60% |
| | enzyme), | | | (Microtubule- | |
| | increasing | | | associated | |
| | cAMP | | | protein 2) | |
| | concentration | | 5 d | NeuN | 30%-50% |
| | | | | (Neuronal nuclei) | |
| | Activating | 8-Bromo-cAMP | 2 d | TUJ1 | 50%-60% |
| | PKA/CREB | (PKA/CREB | 2 d | MAP2 | 30%-40% |
| | | activator): 5 μM | 5 d | NeuN | 15%-25% |
| | | to 500 μM | | | |
| | Inhibiting | SP600125 (JNK | 2 d | TUJ1 | 40%-50% |
| | JNK | inhibitor): 1 μM to | 2 d | MAP2 | 20%-30% |
| | | 100 μM | 5 d | NeuN | 30%-40% |
| | Inhibiting | LDN193189 (BMP | 2 d | TUJ1 | 60%-70% |
| | ALK2/3 | inhibitor): 0.25 μM | 2 d | MAP2 | 50%-60% |
| | | to 50 μM | 5 d | NeuN | 20%-30% |
| | Inhibiting | Dorsomorphin | 2 d | TUJ1 | 40%-50% |
| | AMPK | (AMPK inhibitor): | 2 d | MAP2 | 30%-40% |
| | | 1 μM to 100 μM | 5 d | NeuN | 15%-25% |
| | Increasing | cAMP compound | 2 d | TUJ1 | 75%-85% |
| | cAMP | and analog: 0.1 mM | 2 d | MAP2 | 50%-60% |
| | concentration | to 100 mM | 5 d | NeuN | 40%-50% |
| | Inhibiting | SB203580 (P38 | 2 d | TUJ1 | 40%-50% |
| | P38 | inhibitor): 0.05 μM | 2 d | MAP2 | 30%-40% |
| | | to 50 μM | 5 d | NeuN | 20%-30% |

| | | | | | Cell |
|---|---|---|---|---|---|
| | Action | | Induction | Neuron-specific | positive |
| Treatment | site | Action effect | time | protein | rate |
| Gene | CREB | Overexpression/ | 2 d | TUJ1 | 60%-70% |
| overexpression/ | | activation of | 2 d | MAP2 | 45%-50% |
| knock out | | CREB expression | 5 d | NeuN | 30%-40% |
| | JNK | Knockout/inhibition | 2 d | TUJ1 | 45%-50% |
| | | of JNK | 2 d | MAP2 | 30%-40% |
| | | expression | 5 d | NeuN | 30%-40% |
| | PKA | Overexpression/ | 2 d | TUJ1 | 40%-50% |
| | | activation of | 2 d | MAP2 | 30%-40% |
| | | PKA expression | 5 d | NeuN | 10%-20% |

TABLE 2
Small molecule compounds at each action site

| Action | Activator/ | |
|---|---|---|
| site | inhibitor | Small molecule compounds and concentration range |
| PKA | Activator | 1. Forskolin(Colforsin): 1 μM-100 μM |
| | | 2. 8-Bromo-cAMP: 5 μM-500 μM |
| | | 3. Dibutyryl-cAMP (Bucladesine): 5 μM-500 μM |
| cAMP | Activator | 1. Pure cAMP compound: 0.1 mM to 100 mM |
| | | 2. cAMP analog: 0.1 mM to 100 mM |
| ALK2 | Inhibitor | 1. DMH1: 0.5 μM-50 μM |
| | | 2. K02288: 0.5 μM-50 μM |
| | | 3. LDN-212854: 0.01 μM-10 μM |
| | | 4. LDN-193189: 0.25 μM-50 μM |
| | | 5. LDN-193189 HCl: 0.25 μM-50 μM |
| | | 6. ML347: 0.1 μM-100 μM |
| ALK3 | | 1. LDN-193189 HCl: 0.25 μM-50 μM |
| | | 2. LDN-193189: 0.25 μM-50 μM |
| | | 3. K02288: 0.01 μM-10 μM |
| | | 4. LDN-212854: 0.01 μM-10 μM |
| | | 5. ML347: 0.1 μM-100 μM |
| JNK | Inhibitor | 1. JNK Inhibitor IX: 0.01 μM-100 μM |
| | | 2. JNK Inhibitor VIII: 0.01 μM-100 μM |
| | | 3. JNK-IN-8: 0.01 μM-100 μM |
| | | 4. Tanzisertib(CC-930): 0.1 μM-100 μM |
| | | 5. SP600125: 1 μM-100 μM |
| | | 6. Doramapimod(BIRB796): 1 μM-50 μM |
| | | 7. Metformin HCl: 10 μM-10 mM |
| | | 8. Loureirin B: 2 μM-300 μM |
| | | 9. Bentamapimod(AS602801): 0.01 μM-100 μM |
| | | 10. Ginsenoside Re: 0.1 μM-100 μM |
| | | 11. BI-78D3: 1 nM-10 μM |
| | | 12. CC-401 Hydrochloride: 0.01 μM-100 μM |
| | | 13. Falcarindiol: 0.01 μM-100 μM |
| | | 14. Cucurbitacin IIb: 0.01 μM-100 μM |
| | | 15. Trans-Zeatin: 1 μM-1 nM |
| | | 16. Urolithin B: 0.01 μM-100 μM |
| | | 17. IQ-1S: 0.01 μM-100 μM |
| | | 18. IQ 3: 0.01 μM-100 μM |
| | | 19. DB07268: 0.01 μM-100 μM |
| P38 | Inhibitor | 1. SB203580: 0.05 μM-50 μM |
| | | 2. Doramapimod (BIRB 796): 0.01 μM-100 μM |
| | | 3. SB202190 (FHPI): 0.1 μM-100 μM |
| | | 4. Ralimetinib dimesylate: 0.01 μM-100 μM |
| | | 5. VX-702: 0.01 μM-100 μM |
| | | 6. PH-797804: 0.01 μM-100 μM |
| | | 7. Neflamapimod (VX-745): 10 nM-100 μM |
| | | 8. TAK-715: 0.01 μM-100 μM |
| | | 9. Mulberroside A: 0.01 μM-100 μM |
| | | 10. SD 0006: 0.01 μM-100 μM |
| | | 11. Trans-Zeatin: 1 μM-10 nM |
| | | 12. SB239063: 0.01 μM-100 μM |
| | | 13. BMS-582949: 0.01 μM-100 μM |
| | | 14. ML141: 1 μM-100 μM |
| | | 15. 3′-Hydroxypterostilbene: 0.1 μM-1 mM |
| | | 16. Praeruptorin A: 0.01 μM-100 μM |
| | | 17. Pamapimod: 1 μM-1 nM |
| | | 18. Skepinone-L: 0.01 μM-100 μM |
| | | 19. TA-02: 0.01 μM-100 μM |
| | | 20. Losmapimod(GW856553X): 0.01 μM-100 μM |
| | | 21. UM-164: 0.1 μM-10 μM |
| | | 22. AUDA: 0.01 μM-100 μM |
| | | 23. PD169316: 0.01 μM-100 μM |
| | | 24. Pexmetinib (ARRY-614): 0.01 μM-100 μM |
| AMPK | Inhibitor | 1. Doxorubicin(Adriamycin)HCl: 1 nM-100 μM |
| | | 2. GSK690693: 1 nM-100 μM |
| | | 3. Dorsomorphin (Compound C) 2HCl: |
| | | 1 μM-100 μM |
| | | 4. STO-609: 0.01 μM-100 μM |
| | | 5. EB-3D: 0.01 μM-100 μM |
| | | 6. Dorsomorphin (Compound C): 1 μM-100 μM |

II. The neuronal cell maturation medium includes DMEM/F12 and Neurobasal in a volume ratio of 1:1, 0.5% N2 (by volume percentage), 1% B27 (by volume percentage), 100 μM cAMP, 20 ng/mL bFGF, 20 ng/mL BDNF, 20 ng/mL GDNF, 20 ng/mL NT3, 100 U/mL penicillin, and 0.1 mg/mL streptomycin.
III. The neuronal cell medium includes DMEM/F12 and Neurobasal in a volume ratio of 1:1, 0.5% N2 (by volume percentage), 1% B27 (by volume percentage), 20 ng/mL bFGF, 20 ng/mL BDNF, 20 ng/mL GDNF, 20 ng/mL NT3, 100 U/mL penicillin, and 0.1 mg/mL streptomycin.
IV. Induction method:
1) inoculating the somatic cells into a 60 mm cell culture dish at a density of 5×10⁵ cells; adding a high-glucose DMEM+10% FBS (fibroblast medium/FM), and conducting culture overnight in an incubator at 37° C. and a humidity of 95% with 5% carbon dioxide; conducting induction culture with the neuronal cell induction medium for 48 h to obtain chemical-induced neuronal cells (CiNCs); and
2) replacing the neuronal cell induction medium with a neuronal cell maturation medium to continue promoting further maturation of the CiNCs, and replacing the neuronal cell maturation medium with a neuronal cell medium after 72 h for long-term culture.

### Example 1

Human skin fibroblasts (BJ) had been successfully reprogrammed into functional neuronal cells using the induced reprogramming method.

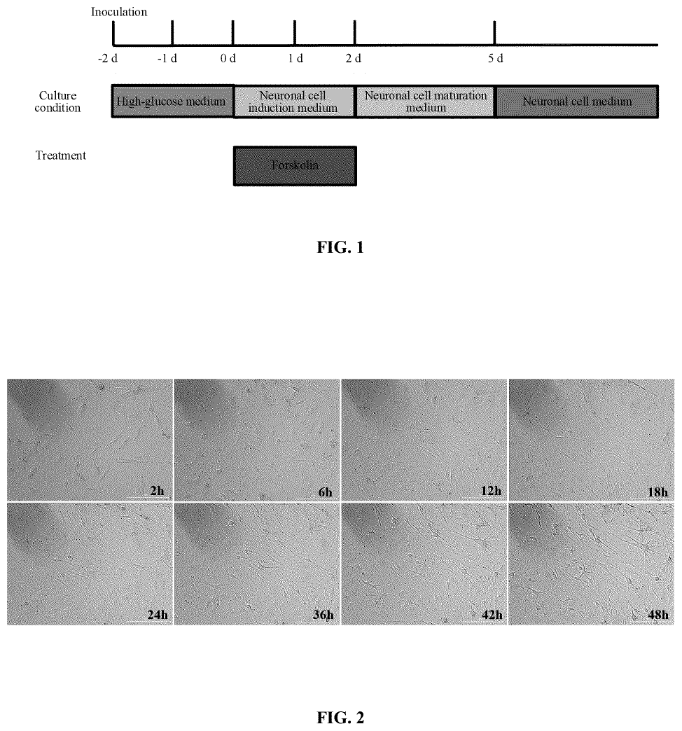

The overall experimental induction was shown in FIG. 1.

Specific operations were as follows:

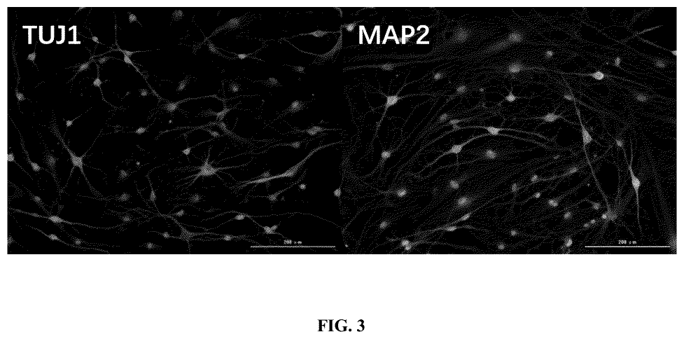

Human skin fibroblasts were inoculated into a 60 mm cell culture dish at a density of 5×10⁵, and a neuronal cell induction medium (N2B27+KSR+10 μM Forskolin) was replaced within 24 h of inoculation, and cultured in a 37° C., 5% CO₂ incubator for 48 h. The morphological changes of cells during induction were shown in FIG. 2, and the CiNCs with a TUJ1 positive rate of 80% were obtained 48 h after induction.

After 48 h of induction, the induction medium was replaced with a neuronal cell maturation medium to continue promoting further maturation of the CiNCs, and the neuronal cell maturation medium was replaced with a neuronal cell medium after 72 h for long-term culture.

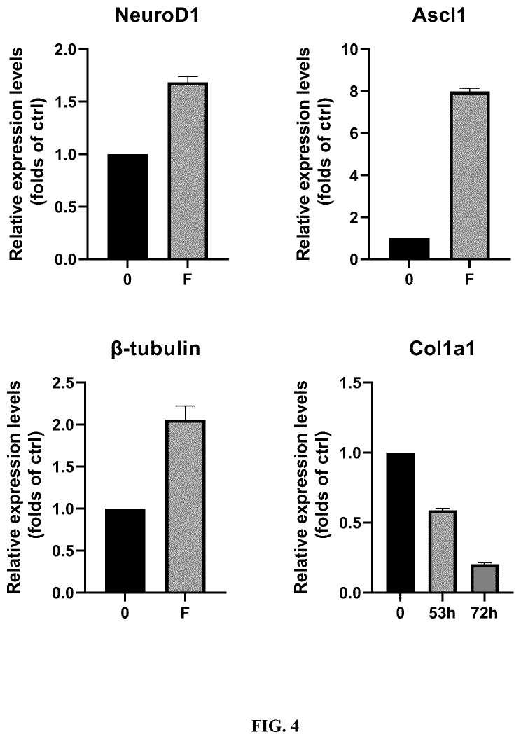

Immunofluorescence detection of neuronal cell marker antigens was conducted on human skin fibroblasts and cells induced by neuronal cell induction (N2B27+10 μM Forskolin) for 48 h. The method included the following specific steps: human skin fibroblasts and F-48h cells in a culture plate were fixated with 4% paraformaldehyde (PFA) at room temperature for 30 min; the cells were washed with a blocking solution three times, 5 min in each time; the cells were permeabilized by 1% TritonX-100 for 15 min at room temperature; the cells were washed with the blocking solution three times again; non-specific sites were blocked with 5% donkey serum, and then blocked for 2 h at room temperature; the cells were washed three times with TBP (Tritonx-Bovine albumin-Phosphate Buffer Saline) for 5 min in each time; a primary antibody was added for incubation at 4° C. overnight; on a next day, the culture plate was placed at room temperature, rewarmed for 20 min, and then washed with TBP three times for 5 min in each time, a secondary antibody and a Hoechst mixture were added in the dark, and incubated at room temperature for 1 h; after washing three times with a TBP solution, fluorescence microscope observation and photographing experiment were conducted. Immunofluorescence staining results (FIG. 3) showed that the CiNCs (F-48 h) expressed the neuronal cell marker antigens, TUJ1 and MAP2, while the human skin fibroblasts did not express same.

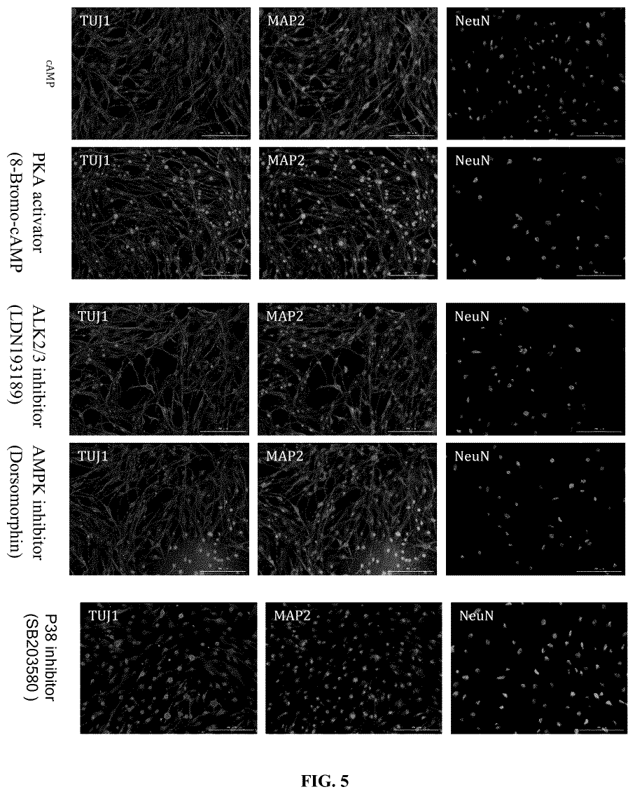

The expression of neuronal cell marker genes was detected by quantitative PCR (qPCR). The specific operation steps were as follows: (1) total RNA extraction: the medium was discarded, cells were washed three times with PBS, and lysed on ice for 5 min with 1 ml of pre-cooled TRIZOL; 200 μL of chloroform was added, shaken vigorously for 15 sec, and placed on ice for 5 min; the cells were centrifuge at 12000 r/min for 15 min at 4° C.; an upper aqueous phase was transferred to pre-cooled isopropanol, inverted and mixed, and placed on ice for 5 min; the cells were centrifuged at 12000 r/min for 10 min at 4° C.; a supernatant was discarded, 1 mL of pre-chilled 75% ethanol was added, RNA was suspended by flicking a bottom of the tube with fingertips, the RNA and the tube wall were washed thoroughly, and centrifuged at 7500 r/min at 4° C. for 8 min; a supernatant was discarded, when the precipitate was translucent, an appropriate amount of DEPC (diethypyrocarbonate) water was added to completely dissolve the RNA, 1 μL of the RNA was collected for purity and integrity testing, and the rest was reverse-transcribed or frozen in a −80° C. refrigerator. (2) cDNA template preparation. A Vazyme R223-01 synthesis kit was used according to the instructions. (3) Fluorescence quantitative PCR. A Vazyme Q711-02/03 reagent was used according to the instructions. The results of qPCR (FIG. 4) showed that compared with human skin fibroblasts (F-0 h), the CiNCs (F-48 h) highly expressed neuronal cell-related marker genes such as NeuroD1, tubulin, and Ascl1 , and fibroblast marker gene Col1A1 had a significantly down-regulated expression level.

### Example 2

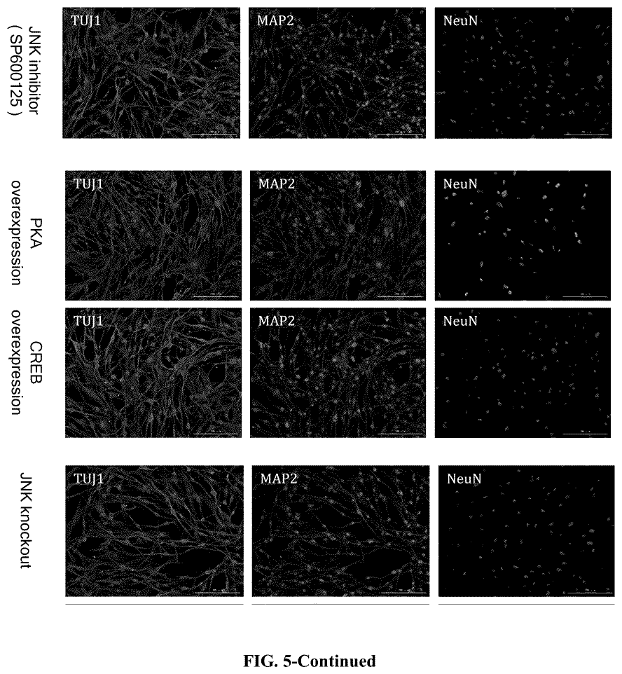

This example differed from Example 1 in that the neuronal cell induction medium was N2B27+KSR+10 mM cAMP. Immunofluorescence assay showed that the CiNCs expressed neuronal cell marker antigens TUJ1, MAP2 and NeuN (FIG. 5).

### Example 3

This example differed from Example 1 in that the neuronal cell induction medium was N2B27+KSR+10 μM 8-Bromo-cAMP. Immunofluorescence assay showed that the CiNCs expressed neuronal cell marker antigens TUJ1, MAP2 and NeuN (FIG. 5).

### Example 4

This example differed from Example 1 in that the neuronal cell induction medium was N2B27+KSR+10 μM Dorsomorphin. Immunofluorescence assay showed that the CiNCs expressed neuronal cell marker antigens TUJ1, MAP2 and NeuN (FIG. 5).

### Example 5

This example differed from Example 1 in that the neuronal cell induction medium was N2B27+KSR+5 μM LDN193189. Immunofluorescence assay showed that the CiNCs expressed neuronal cell marker antigens TUJ1, MAP2 and NeuN (FIG. 5).

### Example 6

This example differed from Example 1 in that the neuronal cell induction medium was N2B27+KSR+5 μMSB203580. Immunofluorescence assay showed that the CiNCs expressed neuronal cell marker antigens TUJ1, MAP2 and NeuN (FIG. 5).

### Example 7

This example differed from Example 1 in that the neuronal cell induction medium was N2B27+KSR+1011M SP600125.Immunofluorescence assay showed that the CiNCs expressed neuronal cell marker antigens TUJ1, MAP2 and NeuN (FIG. 5).

### Example 8

An overexpression recombinant plasmid of a PKA gene (pLVX-PKA-IRES) was constructed. RNA was extracted from cells with high expression of the target gene (PKA) by a Trizol method, and after reverse transcription, primers were designed, and a complete coding region sequence of PKA gene was amplified by PCR using cDNA as a template. PCR products were subjected to agarose electrophoresis, and gel pieces corresponding to the PKA gene fragments were excised for gel recovery. After gel recovery, a DNA concentration was measured to obtain a target gene fragment. Double-enzyme digestion with restriction endonucleases was conducted on an overexpression empty vector (pLVX-IRES) plasmid; after the digestion, the plasmid was subjected to agarose electrophoresis and gel recovery, a concentration of the gel recovered product was determined, and the product was subjected to homologous recombination with the PKA gene fragment. The recombinant product was then transformed into E. coli T1 competent cells, spread on an LB solid plate medium containing ampicillin (Amp⁺), and incubated in a 37° C. incubator for 12 h to 16 h. A single clone was selected and continued to be incubated in a Luria-Bertani (LB) liquid medium containing Amp⁺. The bacterial solution was identified by colony PCR, and the correctly identified bacterial solution was selected to extract the plasmid with a plasmid extraction kit for further enzyme digestion identification. The correctly identified plasmid was sent to a company for sequencing. A bacterial solution corresponding to the correctly-sequenced plasmid was expanded and cultured, and a recombinant overexpression plasmid (pLVX-PKA-IRES) was extracted with an endotoxin-free plasmid extraction kit.

Virus packaging and cell infection-induced neuronal transdifferentiation were conducted. HEK (human embryonic kidney)-293T cells were recovered and placed in a 37° C., 5% CO₂ incubator; when the cell confluence reached 50% to 60%, a Lipofectamine™3000 reagent was diluted by a lipofection method with a serum-free DMEM medium, and mixed well. The DNA (recombinant overexpression plasmid pLVX-PKA-IRES, viral packaging plasmid NRF, and viral envelope plasmid Vesicular stomatitis virus-G (VSVG) in a ration of 5:3:2) was diluted with the serum-free DMEM medium, a DNA premix was prepared and added with a P3000™ reagent, and mixed well. The diluted DNA was added to the diluted Lipofectamine™3000 reagent (at a ratio of Lip3000:DNA=2.5:1). After incubation at room temperature for 10 min, a DNA-liposome complex was co-transfected into the HEK-293T cells, cultured at 37° C., 5% CO₂; and a virus supernatant was collected after 48 h to 72 h, centrifuged at 4° C., 2000 r/min for 10 min, and filtered with a 0.45 μm filter. The human skin fibroblasts (5×10⁵) were directly infected with the filtered virus solution and a culture medium at a mixing ratio of 1:1, transferred to a medium (N2B27+KSR) after 2 d, and incubated in a 37° C., 5% CO₂ incubator for 2 d. The neuronal cell maturation medium was changed, and the cells were continued to incubate for 3 d to further promote the maturation of CiNCs. The cells were transferred to a neuronal cell medium for long-term culture.

Finally, the validation of CiNCs was conducted. Immunofluorescence detection of neuronal cell marker antigens was conducted on the above CiNCs. The method included the following specific steps: human skin fibroblasts and F-48 h cells in a culture plate were fixated with 4% paraformaldehyde (PFA) at room temperature for 30 min; the cells were washed with a blocking solution three times, 5 min in each time; the cells were permeabilized by 1% TritonX-100 for 15 min at room temperature; the cells were washed with the blocking solution three times again; non-specific sites were blocked with 5% donkey serum, and then blocked for 2 h at room temperature; the cells were washed three times with TBP (Tritonx-BSA-PBS) for 5 min in each time; a primary antibody was added for incubation at 4° C. overnight; on a next day, the culture plate was placed at room temperature, rewarmed for 20 min, and then washed with TBP three times for 5 min in each time, a secondary antibody and a Hoechst mixture were added in the dark, and incubated at room temperature for 1 h; after washing three times with a TBP solution, fluorescence microscope observation and photographing experiment were conducted. Immunofluorescence staining results (FIG. 5) showed that the CiNCs expressed the neuronal cell marker antigens, TUJ1, MAP2, and NeuN.

### Example 9

This example differed from Example 8 in that the overexpressed gene was CREB. Immunofluorescence assay showed that the CiNCs expressed neuronal cell marker antigens TUJ1, MAP2 and NeuN (FIG. 5).

### Example 10

A JNK gene-knockout recombinant plasmid (U6-sgJNK-EF1a-Cas9-FLAG-P2A-puro) was constructed. The exons of the JNK gene were selected, and targeted sgJNK was designed using a sgRNA design tool of the MIT Zhang Feng laboratory, and a sticky end of the restriction endonuclease was added to a primer of the sgJNK to facilitate the ligation with an empty vector (U6-sgRNA-EF1a-Cas9-FLAG-P2A-puro). A dry powder of primers synthesized by the company was diluted with ddH₂O to a working concentration, an annealing system was configured to conduct annealing. The empty vector was linearized by the restriction endonuclease, inserted into the sgJNK, and transformed into competent E. coli after ligation, spread on an LB solid plate medium containing ampicillin (Amp⁺), and incubated in a 37° C. incubator for 12 h to 16 h. A single clone was selected and continued to be incubated in an LB liquid medium containing Amp⁺. The bacterial solution was identified by colony PCR, the correctly identified bacterial solution was selected to extract the plasmid with a plasmid extraction kit for further enzyme digestion identification, and the correctly-identified plasmid was sent to the company for sequencing. A bacterial solution corresponding to the correctly-sequenced plasmid was expanded and cultured, and a plasmid containing an original sgJNK expression copy was extracted with an endotoxin-free plasmid extraction kit for transfection of cells.

The packaging and cell infection of JNK gene-knockout virus were conducted. HEK-293T cells were recovered and placed in a 37° C., 5% CO₂ incubator; when the cell confluence reached 50% to 60%, a Lipofectamine™3000 reagent was diluted by a lipofection method with a serum-free DMEM medium, and mixed well. The DNA (Cas9 plasmid, viral packaging plasmid psPAX2 and pMDG2 had a ratio of 5:3:2) was diluted with the serum-free DMEM medium, a DNA premix was prepared and added with a P3000™ reagent, and mixed well. The diluted DNA was added to the diluted Lipofectamine™3000 reagent (at a ratio of Lip3000:DNA=2.5:1). After incubation at room temperature for 10 min, a DNA-liposome complex was co-transfected into the HEK-293T cells, cultured at 37° C., 5% CO₂; and a virus supernatant was collected after 48 h to 72 h, centrifuged at 4° C., 2000 r/min for 10 min, and filtered with a 0.45 μm filter. The human skin fibroblasts (5×10⁵) were directly infected with the filtered virus solution and a culture medium at a mixing ratio of 1:1, transferred to a medium (N2B27+KSR) after 2 d, and incubated in a 37° C., 5% CO₂ incubator for 2 d. The neuronal cell maturation medium was changed, and the cells were continued to incubate for 3 d to further promote the maturation of CiNCs. The cells were transferred to a neuronal cell medium for long-term culture.

Finally, the validation of CiNCs was conducted. Immunofluorescence detection of neuronal cell marker antigens was conducted on the above CiNCs. The method included the following specific steps: human skin fibroblasts and F-48 h cells in a culture plate were fixated with 4% paraformaldehyde (PFA) at room temperature for 30 min; the cells were washed with a blocking solution three times, 5 min in each time; the cells were permeabilized by 1% TritonX-100 for 15 min at room temperature; the cells were washed with the blocking solution three times again; non-specific sites were blocked with 5% donkey serum, and then blocked for 2 h at room temperature; the cells were washed three times with TBP (Tritonx-BSA-PBS) for 5 min in each time; a primary antibody was added for incubation at 4° C. overnight; on a next day, the culture plate was placed at room temperature, rewarmed for 20 min, and then washed with TBP three times for 5 min in each time, a secondary antibody and a Hoechst mixture were added in the dark, and incubated at room temperature for 1 h; after washing three times with a TBP solution, fluorescence microscope observation and photographing experiment were conducted. Immunofluorescence staining results (FIG. 5) showed that the CiNCs expressed the neuronal cell marker antigens, TUJ1, MAP2, and NeuN.

Therefore, results of the above examples prove that a single small molecule compound, or gene knockout/overexpression of any site on the regulatory signaling pathways (cAMP, PKA, CREB, AMPK, ALK2, ALK3, P38, and JNK) can induce high-efficiency reprogramming of in human cells into neuronal cells.

The above described are merely preferred embodiments of the present disclosure, and not intended to limit the present disclosure. Any modifications, equivalent replacements and improvements made within the spirit and principle of the present disclosure should all fall within the scope of protection of the present disclosure.

1. A method for efficiently inducing reprogramming of human cells into neuronal cells, comprising: up-regulating an expression of an action site that is selected from a group consisting of protein kinase A (PKA) and Cyclic Adenosine Monophosphate response-element binding protein (CREB) by using a single small molecule compound.

2. The method according to claim 1, wherein the single small molecule compound comprises one or more of a PKA activator, and a CREB activator.

3. An induction medium for efficiently inducing reprogramming of human cells into neuronal cells, comprising a basal solution, KnockOut Serum Replacement (KSR), and a small molecule compound, wherein preferably, the small molecule compound comprises one or more of a cAMP activator, cAMP, a cAMP analog, a PKA activator, a CREB activator, an AMPK inhibitor, an ALK2 inhibitor, an ALK3 inhibitor, a P38 inhibitor, and a JNK inhibitor.

4. The induction medium for efficiently inducing reprogramming of human cells into neuronal cells according to claim 3, wherein the small molecule compound comprises Forskolin, 8-Bromo-cAMP, LDN193189, the cAMP, the cAMP analog, SP600125, SB203580, and Dorsomorphin, with concentrations in a final medium sequentially as follows: 0 μM to 100 μM, 0 μM to 500 μM, 0 μM to 25 μM, 0 nM to 10 mM, 0 mM to 10 mM, 0 μM to 10 μM, 0 μM to 5 μM, and 0 μM to 100 μM, respectively, preferably 5 μM to 20 μM, 5 μM to 50 μM, 0.5 μM to 5 μM, 0.5 mM to 5 mM, 0.5 mM to 5 mM, 0.5 μM to 5 μM, 0.1 μM to 2.5 μM, and 0.5 μM to 20 μM, respectively, more preferably 10 μM, 50 μM, 2.5 μM, 1 mM, 1 mM, 1 μM, 0.5 μM, and 10 μM, respectively; and the concentration of the above substances are not all 0.

5. The induction medium for efficiently inducing reprogramming of human cells into neuronal cells according to claim 3, wherein the basal solution and the KSR have a volume ratio of 80:20; and preferably, the basal solution is N2B27, comprising Knockout Dulbecco's Modified Eagle Medium: F-12 (DMEM/F12), N-2 Supplement (N2, 100×), Neurobasal, B-27 Supplement (B27, 50×), and Glutamine (100×) with a volume ratio of 99:1:97:2:1.

6. Use of the induction medium according to claim 3 in in-vitro and in-vivo induction of reprogramming of somatic cells into neuronal cells.

7. A method for inducing reprogramming of somatic cells into neuronal cells in vitro using an induction medium, comprising the following steps:
inoculating the somatic cells into a petri dish; adding a high-glucose dulbecco's modified eagle medium and 10% fetal bovine serum medium (DMEM+10% FBS), and conducting culture overnight in an incubator at 37° C. and a humidity of 95% with 5% carbon dioxide; conducting induction culture with the induction medium according to any one of claims 3 to 5 for 48 h to obtain chemical-induced neuronal cells (CiNCs); and
replacing the induction medium with a neuronal cell maturation medium to continue promoting further maturation of the CiNCs, and replacing the neuronal cell maturation medium with a neuronal cell medium after 72 h for long-term culture.

8. The method according to claim 7, wherein the neuronal cell maturation medium comprises DMEM/F12 and Neurobasal in a volume ratio of 1:1, 0.5% N2 (by volume percentage), 1% B27 (by volume percentage), 100 μM cAMP, 20 ng/mL basic fibroblast growth factor (bFGF), 20 ng/mL brain-derived neurotrophic factor (BDNF), 20 ng/mL glialcellline-derivedneurotrophicfactor (GDNF), 20 ng/mL Neurotrophin 3 (NT3), 100 U/mL penicillin, and 0.1 mg/mL streptomycin.

9. The method according to claim 7, wherein the neuronal cell medium comprises DMEM/F12 and Neurobasal in a volume ratio of 1:1, 0.5% N2 (by volume percentage), 1% B27 (by volume percentage), 20 ng/mL bFGF, 20 ng/mL BDNF, 20 ng/mL GDNF, 20 ng/mL NT3, 100 U/mL penicillin, and 0.1 mg/mL streptomycin.

10. The method according to claim 1, wherein the human cells are skin fibroblasts, granulosa cells, or astrocytes that are derived from a human being.

11. The induction medium according to claim 3, wherein the somatic cells are skin fibroblasts, granulosa cells, or astrocytes that are derived from a human being, a monkey, or a mouse.

12. The use according to claim 6, wherein the somatic cells are skin fibroblasts, granulosa cells, or astrocytes that are derived from a human being, a monkey, or a mouse.

13. The method according to claim 7, wherein the somatic cells are skin fibroblasts, granulosa cells, or astrocytes that are derived from a human being, a monkey, or a mouse.
